(19) United States
(12) Patent Application Publication
(10) Pub. No.: US 2010/0010538 A1
(43) Pub. Date: Jan. 14, 2010

(54) RESHAPING THE MITRAL VALVE OF A HEART

(75) Inventors: Mark S. Juravic, SAN FRANCISCO, CA (US); Michael C. Stewart, MILPITAS, CA (US)

Correspondence Address: LAW OFFICE OF ALAN W. CANNON, 942 MESA OAK COURT, SUNNYVALE, CA, 94086, US

(73) Assignee: MAQUET CARDIOVASCULAR LLC, SAN JOSE, CA (US)

(21) Appl. No.: 12/172,069

(22) Filed: Jul. 11, 2008

(51) Int. Cl. A61B 17/06; A61F 2/24; A61B 19/00

(52) U.S. Cl. 606/228; 623/2.11; 606/232; 128/898

(57) ABSTRACT

Assemblies and methods for reshaping a portion of a heart, such as the mitral valve and/or the ventricle, are disclosed. The assemblies include a plurality of tissue anchors and a connecting member extending between the plurality of tissue anchors. The tissue anchors and the connecting member act to reshape a portion of the heart, without the need to gain access to the interior of the heart.

### TECHNICAL FIELD

The disclosure is directed to medical devices, assemblies and methods for reshaping a portion of a heart. More particularly, the disclosure is directed to devices, assemblies and methods for reshaping the mitral valve and/or ventricle of a heart in order to reduce or eliminate retrograde blood flow through the mitral valve of a heart.

### BACKGROUND

The mitral valve is located between the left atrium and the left ventricle of the heart. During normal operation, the mitral valve opens during diastole, allowing blood to flow from the left atrium into the left ventricle. During systole, the mitral valve closes, causing high pressure blood to exit the left ventricle through the aorta. Mitral valve regurgitation is a cardiac condition in which the posterior leaflet of the mitral valve does not fully contact the anterior leaflet of the valve during systole, thus a gap remains between the leaflets of the mitral valve during systole. The gap remaining between the leaflets allows retrograde blood flow to pass from the left ventricle into the left atrium through the mitral valve. Thus, mitral regurgitation reduces the volume of blood pumped out of the heart to the aorta during each cardiac cycle, thus reducing the efficiency of the heart. Mitral regurgitation may exist for any of several reasons, including congenital malformations of the valve, ischemic disease, or effects of cardiomyopathy, such as dilated (congestive) cardiomyopathy (i.e., enlarging of the heart).

Conventional techniques for treating dysfunctions of the mitral valve typically include highly invasive, open heart surgical procedures in order to replace or repair the dysfunctioning mitral valve. Some surgical procedures include the implantation of a replacement valve (e.g., animal valve or artificial mechanical valve). Other techniques include the use of annuloplasty rings which are surgically placed around the annulus of the mitral valve within the chamber of the heart and sutured into place. The presence of the annuloplasty ring alters the geometry of the annulus of the mitral valve in order to improve coaptation of the leaflets of the valve. Another surgical technique which requires accessing one or more chambers of the heart is leaflet coaptation. Leaflet coaptation (e.g., Alfieri edge-to-edge repair) is a surgical procedure in which the valve leaflets are sutured together (e.g., bow-tie suture) to improve coaptation of the leaflets. A further surgical technique includes extending a tensioning cord across a chamber of the heart to alter the geometry of the heart chamber. The tensioning cord, which extends through a chamber of the heart, and thus is in contact with blood in the heart chamber, pulls opposing walls of the heart toward one another to reduce heart wall tension and/or reposition the papillary muscles within the chamber. These techniques typically require opening the heart and/or entering one or more of the chambers of the heart to gain direct access to the mitral valve.

Therefore, it is desirable to devise a less invasive technique for treating mitral valve regurgitation. Namely, it is desirable to devise a device, assembly and/or method useful in altering and/or reshaping the annulus of the mitral valve and/or the ventricle of a heart without the need to gain access to the interior of the heart.

### SUMMARY

The disclosure is directed to several alternative designs, materials and methods of manufacturing medical device structures and assemblies.

Accordingly, one illustrative embodiment is an assembly for remodeling and/or reshaping a portion of a heart, such as the mitral valve or ventricle of a heart. The assembly includes a plurality of tissue anchors, such as corkscrew anchors. Additionally, a connecting member may be connected to the tissue anchors, such as by extending end portions of the connecting member through eyelets of the tissue anchors.

Another illustrative embodiment is a method of improving the apposition of the valve leaflets of the mitral valve of a heart in order to reduce retrograde blood flow through the mitral valve. The method includes anchoring a first tissue anchor into a wall of the heart from the epicardial surface of the heart and anchoring a second tissue anchor into a wall of the heart from the epicardial surface of the heart. A connecting member, such as a rigid bar, may be extended between the first tissue anchor and the second tissue anchor exterior of the epicardial surface of the heart such that a first end region of the connecting member is attached to the first tissue anchor and a second end region of the connecting member is attached to the second tissue anchor. Placement of the connecting member between the first tissue anchor and the second tissue anchor causes the central region of the connecting member to apply an inward force on the epicardial surface of the heart, the inward force represented by a vector having a magnitude and direction. Additionally, a pair of opposing outward forces act on the heart wall by the first anchor and the second anchor, wherein each of the pair of opposing outward forces are represented by a vector having a magnitude and direction, wherein the sum of the magnitudes of the vectors of the pair of outward forces is equal to the magnitude of the inward force, and wherein the direction of each of the vectors of the pair of outward forces is opposite the direction of the inward force.

Another illustrative embodiment is a method of improving the functioning of the mitral valve of a heart. The method includes securing a first corkscrew anchor into a wall of the heart from the epicardial surface of the heart and securing a second corkscrew anchor into a wall of the heart from the epicardial surface of the heart. A first end region of a rigid member is placed through the eyelet of the first corkscrew anchor and a second end region of the rigid member is placed through the eyelet of the second corkscrew anchor. Placement of the rigid member between the first anchor and the second anchor causes the central region of the rigid member to apply an inward force on the epicardial surface of the heart, and wherein each of the first corkscrew anchor and the second corkscrew anchor apply an opposing outward force on the wall of the heart.

Yet another illustrative embodiment is a method of reshaping the mitral valve of a heart. The method includes anchoring a first tissue anchor into a wall of the heart from the epicardial surface of the heart at the P1 region of the heart, and anchoring a second tissue anchor into a wall of the heart from the epicardial surface of the heart at the P3 region of the heart. A rigid bar is then extended between the first tissue anchor and the second tissue anchor exterior of the epicardial surface of the heart such that a first end region of the rigid bar is attached to the first tissue anchor and a second end region of the rigid bar is attached to the second tissue anchor. Placement of the rigid bar between the first anchor and the second anchor causes the central region to apply an inward force on the epicardial surface of the heart at the P2 region of the heart, and each of the first anchor and the second anchor apply an opposing outward force on the wall of the heart.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIGS. 1A-1C depict an exemplary tissue anchor useful in reshaping a portion of a heart;

FIG. 2 depicts another illustrative tissue anchor useful in reshaping a portion of a heart;

FIGS. 3A-3B depict yet another illustrative tissue anchor useful in reshaping a portion of a heart;

FIG. 4 illustrates an exemplary connecting member useful in reshaping a portion of a heart;

FIG. 5 depicts another illustrative connecting member useful in reshaping a portion of a heart;

FIG. 6 depicts another illustrative connecting member useful in reshaping a portion of a heart;

FIG. 7 depicts yet another illustrative connecting member useful in reshaping a portion of a heart;

FIGS. 8A-8B illustrate an exemplary tool for fastening a tissue anchor to a wall of a heart;

FIGS. 9A-9B illustrate another exemplary tool for fastening a tissue anchor to a wall of a heart;

FIG. 10 illustrates one possible assembly for reshaping a portion of a heart including a connecting member and a plurality of tissue anchors positioned on the exterior of the heart;

FIG. 11 is an illustrative view representing the placement of the components of the assembly as shown in FIG. 10, in relation to the mitral valve and wall of a heart;

FIG. 12 is a force diagram showing the forces, depicted as vectors, exerted on various portions of the heart and components of the assembly in the illustrative configuration shown in FIG. 11;

FIG. 13 illustrates another possible assembly for reshaping a portion of a heart including a connecting member and a plurality of tissue anchors positioned on the exterior of the heart;

FIG. 14 illustrates yet another possible assembly for reshaping a portion of a heart including a connecting member and a plurality of tissue anchors positioned on the exterior of the heart; and

FIGS. 15, 16A and 16B illustrate another possible assembly for reshaping a portion of a heart including a connecting member and a plurality of tissue anchors positioned on the exterior of the heart.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5). Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

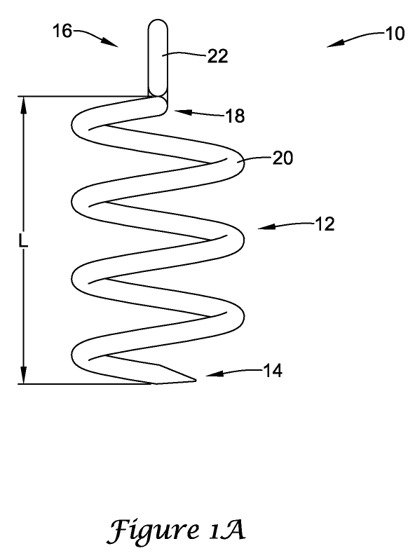

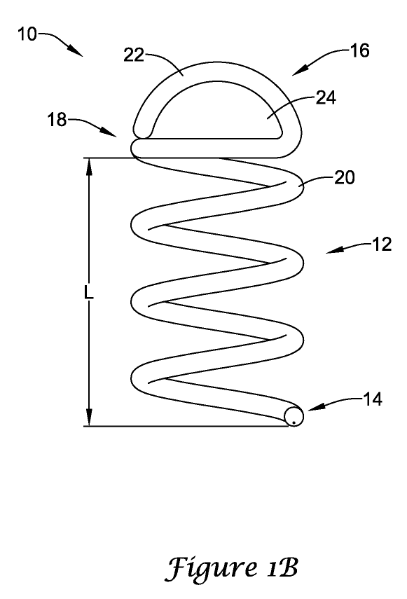

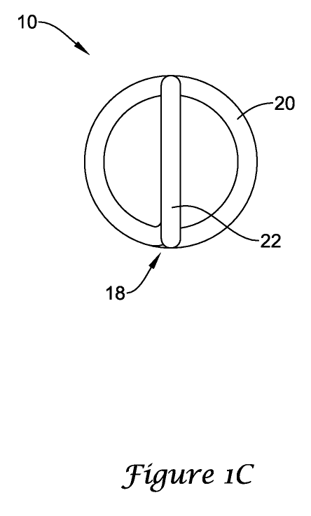

Referring now to FIGS. 1A-1C, there is shown an exemplary tissue anchor 10, for use in reshaping a portion of a heart, such as the mitral valve or ventricle of the heart. The tissue anchor 10 is shown as a corkscrew anchor including a helical portion 12 terminating in a tissue piercing tip 14.

The helical portion 12 of the tissue anchor 10 may have a length L of about 0.5 centimeters to about 2 centimeters, about 0.75 centimeters to about 1.5 centimeters, or about 1 centimeter in some embodiments. In some embodiments, the length L of the helical portion 12 of the tissue anchor 10 may be sized such that the tissue anchor 10 may be anchored to the epicardium layer and/or myocardium layer of a heart from the epicardial surface of the heart, without penetrating the endocardium layer of the heart. In other words, in some embodiments, the tissue anchor 10 may be embedded into the epicardium layer and/or myocardium layer of the heart, but not extend into the endocardium layer and/or may not extend into a chamber of the heart. Thus, the helical portion 12 of the tissue anchor 10 may have a length such that the tissue anchor 10 does not extend entirely through a wall of the heart when the tissue anchor 10 is secured to a wall of the heart.

In some embodiments, the tissue piercing tip 14 of the tissue anchor 10 may be a sharpened tip to facilitate piercing the epicardial surface of a heart. In other embodiments the tissue piercing tip 14 of the tissue anchor 10 may include a hook, barb or other configuration in order to facilitate retention of the tissue anchor 10 in heart tissue.

The end of the helical portion 12 opposite the tissue piercing tip 14 may include an eyelet 16, or other connector means for connecting one or more additional components to the tissue anchor 10. In some embodiments, the eyelet 16 may be formed by bending or forming a portion of the tissue anchor 10 across the diameter of the helical portion 12. For instance, in some embodiments, the tissue anchor 10 may be formed of a single wire filament 20. The wire filament 20 may be helically wound to form the helical portion 12. At the uppermost end 18 of the helical portion 12, the wire filament 20 may be bent in a direction extending across the diameter of the helical portion 12 in an arcuate trajectory. Thus, the wire filament 20 may form an arc shaped portion 22 extending across the diameter of the helical portion 12. An opening 24 defined by the arc shaped portion 22 may be configured to receive another component extending therethrough. Thus the arc shaped portion 22 of the wire filament 20 may, at least in part, form the eyelet 16. In other embodiments, the eyelet 16 may be formed by welding, bonding, or otherwise securing a member, such as an annular member with an opening, to the helical portion 12 of the tissue anchor 10.

The tissue anchor 10 may be formed of any suitable material. For example, the member 12 may be made from a metal, metal alloy, polymer, a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium alloys; combinations thereof; and the like; or any other suitable material.

Some examples of suitable polymers may include fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, polyester, polybutylene terephthalate (PBT), polyethylene terephthalate (PET), polyetheretherketone (PEEK), polyimide (PI), biocompatible polymers, other suitable materials, or mixtures, combinations, copolymers thereof, polymer/metal composites, and the like.

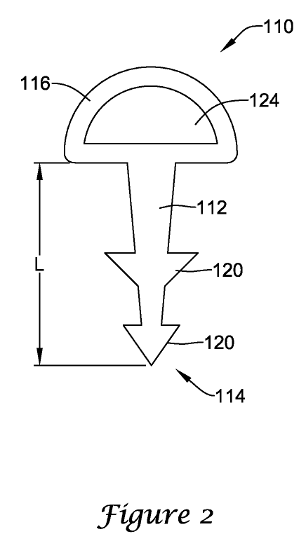

Another tissue anchor 110 is shown in FIG. 2. The tissue anchor 110 may be formed of any desired materials, including those listed above regarding the tissue anchor 10. The tissue anchor 110 may include a shaft 112 terminating in a tissue piercing tip 114. In some embodiments the shaft 112 may taper to a smaller diameter towards the tissue piercing tip 114. In other embodiments, the shaft 112 may have a generally constant diameter. In some embodiments, the tissue piercing tip 114 of the tissue anchor 110 may be a sharpened tip to facilitate piercing the epicardial surface of a heart. The shaft 112 may include one or more, or a plurality of barbs 120 facilitating retention of the tissue anchor 110 in the wall of a heart.

The shaft 112 of the tissue anchor 110 may have a length L of about 0.5 centimeters to about 2 centimeters, about 0.75 centimeters to about 1.5 centimeters, or about 1 centimeter in some embodiments. In some embodiments, the length L of the shaft 112 of the tissue anchor 110 may be sized such that the tissue anchor 110 may be anchored to the epicardium layer and/or myocardium layer of a heart from the epicardial surface of the heart, without penetrating the endocardium layer of the heart. In other words, in some embodiments, the tissue anchor 110 may be embedded into the epicardium layer and/or myocardium layer of the heart, but not extend into the endocardium layer and/or may not extend into a chamber of the heart. Thus, the shaft 112 of the tissue anchor 110 may have a length such that the tissue anchor 110 does not extend entirely through a wall of the heart when the tissue anchor 110 is secured to a wall of the heart.

The shaft 112 of the tissue anchor 110 may extend from an eyelet 116, or other connector means for connecting one or more additional components to the tissue anchor 110. In some embodiments, the eyelet 116 may be a semi-spherical, or hemispherical eyelet. An opening 124 extending through the eyelet 116 may be configured to receive another component extending therethrough. It is noted that in other embodiments, the eyelet 116 may be another desired shape configured to receive another component extending therethrough.

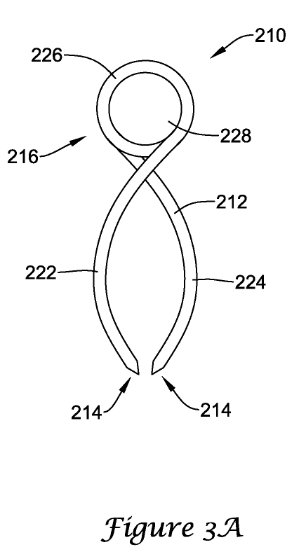

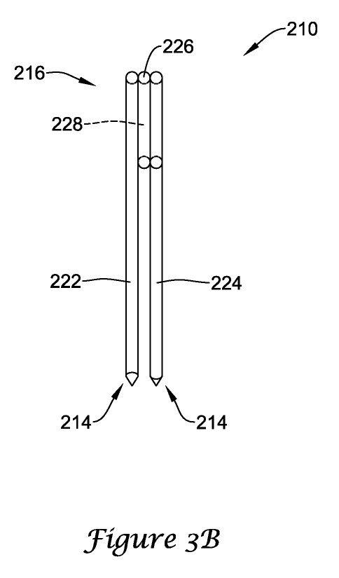

Another tissue anchor 210 is illustrated in FIGS. 3A and 3B. The tissue anchor 210 may be formed of any desired materials, including those listed above regarding the tissue anchor 10. The tissue anchor 210 may include a member 212 having a first arm 222 and a second arm 224 extending from a helical spring, such as a torsion spring 226. In some embodiments, the tissue anchor 210 may be formed of a continuous wire member forming the first arm 222, the torsion spring 226, and the second arm 224. The first arm 222 may terminate at a tissue piercing tip 214 and the second arm 224 may terminate at a tissue piercing tip 214.

The torsion spring 226 may include one, two, three, or more windings of the member 212 wrapped in a helical fashion. The torsion spring 226 may bias the piercing tips 214 of the first arm 222 and the second arm 224 toward one another. In other words, application of an external force which overcomes the biasing force of the torsion spring 226 may urge the first arm 222 away from the second arm 224. However, discontinuance of the external force or reduction of the external force below that of the biasing force of the torsion spring 226 results in the piercing tips 214 of the first arm 222 and the second arm 224 moving toward one another until an equilibrium condition is reached.

Furthermore, the torsion spring 226 may include a central opening 228 extending therethrough, allowing the torsion spring 226 to act as an eyelet 216 which may be configured to receive another component extending through the opening 228 of the torsion spring 226.

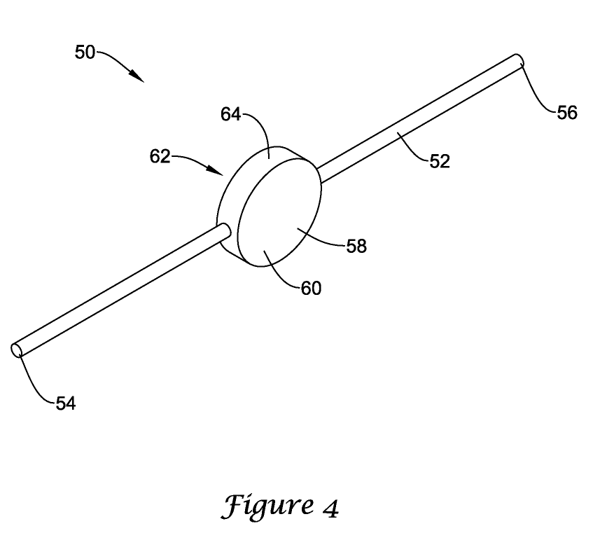

An exemplary connecting member 50 is shown in FIG. 4. The connecting member 50 includes a shaft 52 having a first end 54 and a second end 56. The shaft 52 may be formed of any desired materials, including those listed above with the discussion of the tissue anchor 10. In some embodiments the shaft 52 may be a rigid, non-flexible shaft. In describing the shaft 52 as being rigid, what is meant is the shaft 52 has sufficient rigidity to maintain a desired shape without deformation under normal operating conditions. Thus, application of a typical external force on the rigid shaft 52 will not appreciatively alter the shape of the rigid shaft 52. For example, in some embodiments an external force of 5 Newtons or less, 10 Newtons or less, 15 Newtons or less, 20 Newtons or less, or 25 Newtons or less applied to the rigid shaft 52 would not result in appreciable deflection, deformation or bending of the shaft 52. Furthermore, the shaft 52, unlike a cord or cable, may be capable of withstanding compressive forces without collapsing and/or may be capable of withstanding bending forces without deflection. In some embodiments, the rigid shaft 52 may have a modulus of rigidity of greater than 25 GPa, greater than 30 GPa, greater than 40 GPa, greater than 50 GPa, greater than 60 GPa, greater than 70 GPa, or greater than 80 GPa.

In some embodiments, the shaft 52 may be straight or substantially straight, or in other embodiments, the shaft 52 may be curved or bent into a desired shape. In some embodiments the shaft 52 may have a curvature approximating the curvature of the external curvature of a wall of a heart.

During use, the first end 54 of the shaft 52 may be positioned through an eyelet of a first tissue anchor and/or the second end 56 of the shaft 52 may be positioned through an eyelet of a second tissue anchor.

As shown in FIG. 4, in some embodiments, the connecting member 50 may include a pad 58 connected to the shaft 52, such as a central portion of the shaft 52. For example, in some embodiments, the shaft 52 may extend through the pad 58 such that the pad 58 surrounds a central portion of the shaft 52. However, in other embodiments, the pad 58 may be attached to the shaft 52 in any desired way. The pad 58 may be formed of a polymeric foam material, an ePTFE material, a molded silicone material, a polyester velour, a polypropylene felt, a tight weave polyester, a woven or braided fabric, a non-woven fabric, porous material, a biocompatible material, or other material, as desired. In some embodiments, the material of the pad 58 may promote tissue in-growth on the epicardial surface of the heart and/or provide adequate frictional forces (traction) to hold the pad 58 in contact with the epicardial surface of the heart and prevent migration of the connecting member 50 once positioned on the heart. Tissue in-growth may provide long-term retention of the connecting member 50 in a desired position on the heart and prevent erosion or irritation.

In FIG. 4, the pad 58 is illustrated as a generally disk shape, having a first circular side surface 60, a second circular side surface 62, and a peripheral surface 64 extending between the first side surface 60 and the second side surface 62. It is noted that although the pad 58 is depicted as having a disk shape, in other embodiments the pad 58 may assume any other desired shape, such as square, rectangular, oval, polygonal, irregular, or the like.

In some embodiments, the first side surface 60 and/or the second side surface 62 of the pad 58 may include or be coated with a therapeutic agent, such as a therapeutic agent disclosed later herein. Thus, in some embodiments the surface of the pad 58 which is in contact with the epicardial surface of a heart may include or be coated with a therapeutic agent.

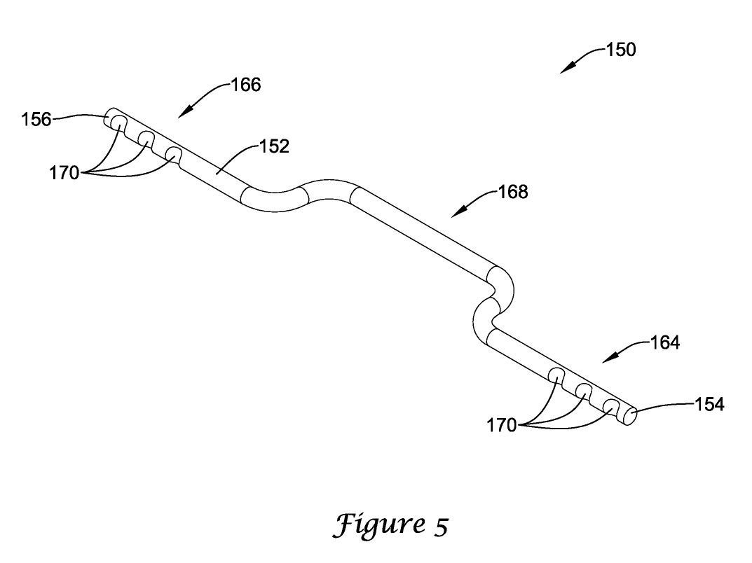

Another connecting member 150 is shown in FIG. 5. The connecting member 150 includes a shaft 152 extending from a first end 154 to a second end 156. The shaft 152 may be formed of any desired materials, including those listed above with the discussion of the tissue anchor 10. In some embodiments the shaft 152 may be a rigid, non-flexible shaft. In describing the shaft 152 as being rigid, what is meant is the shaft 152 has sufficient rigidity to maintain a desired shape without deformation under normal operating conditions. Thus, application of a typical external force on the rigid shaft 152 will not appreciatively alter the shape of the rigid shaft 152. For example, in some embodiments an external force of 5 Newtons or less, 10 Newtons or less, 15 Newtons or less, 20 Newtons or less, or 25 Newtons or less applied to the rigid shaft 152 would not result in appreciable deflection, deformation or bending of the shaft 52. Furthermore, the shaft 152, unlike a cord or cable, may be capable of withstanding compressive forces without collapsing and/or may be capable of withstanding bending forces without deflection. In some embodiments, the rigid shaft 152 may have a modulus of rigidity of greater than 25 GPa, greater than 30 GPa, greater than 40 GPa, greater than 50 GPa, greater than 60 GPa, greater than 70 GPa, or greater than 80 GPa.

The shaft 152 may include a first end portion 164 proximate the first end 154 of the shaft 152, a second end portion 166 proximate the second end 156 of the shaft 152, and a central portion 168 intermediate the first end portion 164 and the second end portion 166. In some embodiments the first end portion 164 of the shaft 152 may be axially aligned with the second end portion 166 of the shaft 152, while the central portion 168 of the shaft 152 may not be axially aligned with the first and second end portions 164/166. Thus, in some embodiments, the central portion 168 of the shaft 152 may be offset from the first end portion 164 and/or the second end portion 166 of the shaft 152. For instance, the shaft 152 may be curved or bent between the first end portion 164 and the central portion 168, and the shaft 152 may be curved or bent between the second end portion 166 and the central portion 168.

During use, the central portion 168 may be placed in contact with the epicardial surface of a heart, whereas the first end portion 164 and/or the second end portion 166 of the shaft 152 may be held above the epicardial surface of a heart with two or more tissue anchors, such as the tissue anchors described above.

During use, the first end 154 of the shaft 152 may be positioned through an eyelet of a first tissue anchor and/or the second end 156 of the shaft 152 may be positioned through an eyelet of a second tissue anchor.

The first end portion 164 may include one or more, or a plurality of notches 170 formed in the shaft 152. The one or more notches 170 may be sized such that a portion of an anchor (as discussed above) may reside in the one or more notches 170. A plurality of notches 170 may allow the position of the connecting member 150 to be adjusted relative to an anchor.

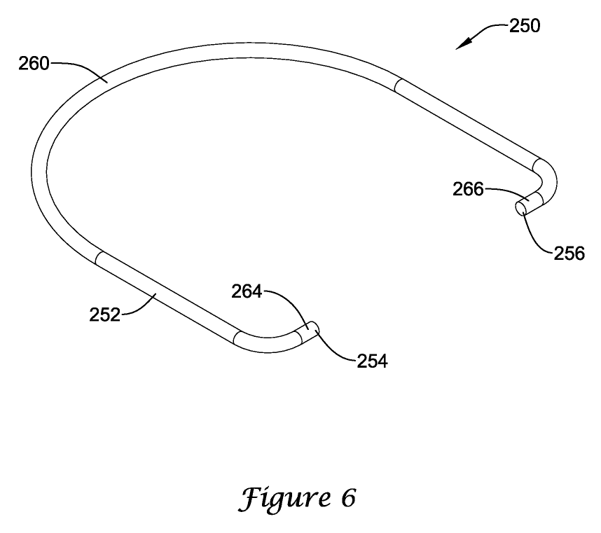

Another connecting member 250 is shown in FIG. 6. The connecting member 250 includes a shaft 252 having a first end 254 and a second end 256. The shaft 252 may be formed of any desired materials, including those listed above with the discussion of the tissue anchor 10. In some embodiments the shaft 252 may be a rigid, non-flexible shaft. In describing the shaft 252 as being rigid, what is meant is the shaft 252 has sufficient rigidity to maintain a desired shape without deformation under normal operating conditions. Thus, application of a typical external force on the rigid shaft 252 will not appreciatively alter the shape of the rigid shaft 252. For example, in some embodiments an external force of 5 Newtons or less, 10 Newtons or less, 15 Newtons or less, 20 Newtons or less, or 25 Newtons or less applied to the rigid shaft 52 would not result in appreciable deflection, deformation or bending of the shaft 252. Furthermore, the shaft 252, unlike a cord or cable, may be capable of withstanding compressive forces without collapsing and/or may be capable of withstanding bending forces without deflection. In some embodiments, the rigid shaft 252 may have a modulus of rigidity of greater than 25 GPa, greater than 30 GPa, greater than 40 GPa, greater than 50 GPa, greater than 60 GPa, greater than 70 GPa, or greater than 80 GPa.

The shaft 252 may be formed or bent into a U-shape, having a central curved portion 260 intermediate a first stub 264 and a second stub 266 of the shaft 252. In some embodiments, the first stub 264 and the second stub 266 of the shaft 252 may be bent toward one another. The first stub 264 may be adjacent the first end 254 of the shaft 252, whereas the second stub 266 may be adjacent the second end 256 of the shaft 252. In some embodiments the first stub 264 and the second stub 266 may be axially aligned with one another. As shown in FIG. 6, the first stub 264 may be turned inward toward the second end 256 of the shaft 252 and/or the second stub 266 may be turned inward toward the first end 254 of the shaft 252. In other embodiments, the first stub 264 and/or the second stub 266 may be turned outward. The first stub 264 and the second stub 266 may be useful in securing the shaft 252 with one or more tissue anchors, such as the tissue anchors described above. For instance, the first stub 264 of the shaft 252 may be positioned through an eyelet of a first tissue anchor and/or the second stub 266 of the shaft 252 may be positioned through an eyelet of a second tissue anchor.

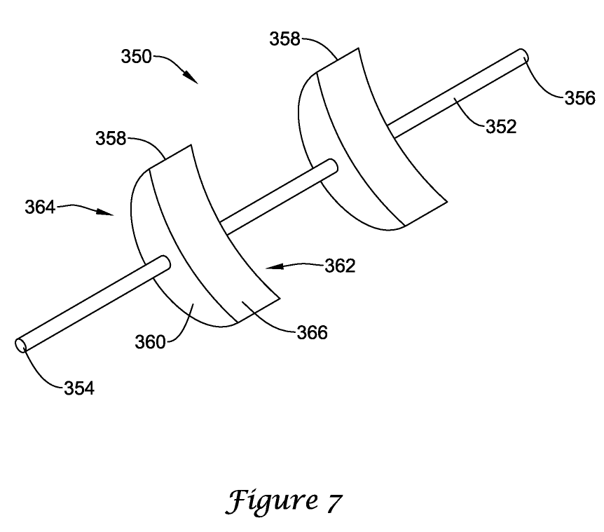

Another connecting member 350 is shown in FIG. 7. The connecting member 350 includes a shaft 352 having a first end 354 and a second end 356. The shaft 352 may be formed of any desired materials, including those listed above with the discussion of the tissue anchor 10. In some embodiments the shaft 352 may be a rigid, non-flexible shaft. In describing the shaft 352 as being rigid, what is meant is the shaft 352 has sufficient rigidity to maintain a desired shape without deformation under normal operating conditions. Thus, application of a typical external force on the rigid shaft 352 will not appreciatively alter the shape of the rigid shaft 352. For example, in some embodiments an external force of 5 Newtons or less, 10 Newtons or less, 15 Newtons or less, 20 Newtons or less, or 25 Newtons or less applied to the rigid shaft 52 would not result in appreciable deflection, deformation or bending of the shaft 352. Furthermore, the shaft 352, unlike a cord or cable, may be capable of withstanding compressive forces without collapsing and/or may be capable of withstanding bending forces without deflection. In some embodiments, the rigid shaft 352 may have a modulus of rigidity of greater than 25 GPa, greater than 30 GPa, greater than 40 GPa, greater than 50 GPa, greater than 60 GPa, greater than 70 GPa, or greater than 80 GPa.

In some embodiments, the shaft 352 may be straight or substantially straight, or in other embodiments, the shaft 352 may be curved or bent into a desired shape. In some embodiments the shaft 352 may have a curvature approximating the curvature of the external curvature of a wall of a heart.

During use, the first end 354 of the shaft 352 may be positioned through an eyelet of a first tissue anchor and/or the second end 356 of the shaft 52 may be positioned through an eyelet of a second tissue anchor.

As shown in FIG. 7, in some embodiments, the connecting member 350 may include one or more, or a plurality of pads 358 connected to the shaft 352, such as a central portion of the shaft 352. For example, in some embodiments, the shaft 352 may extend through the pads 358 such that the pads 358 surround a central portion of the shaft 352. In other embodiments, the pads 358 may be attached to the shaft 352 in any desired way. The pads 358 may be formed of a polymeric foam material, an ePTFE material, a molded silicone material, a polyester velour, a polypropylene felt, a tight weave polyester, a woven or braided fabric, a non-woven fabric, porous material, a biocompatible material, or other material, as desired. In some embodiments, the material of the pads 358 may promote tissue in-growth on the epicardial surface of the heart and/or provide adequate frictional forces (traction) to hold the pads 358 in contact with the epicardial surface of the heart and prevent migration of the connecting member 350 once positioned on the heart. Tissue in-growth may provide long-term retention of the connecting member 350 in a desired position on the heart and prevent erosion or irritation.

In FIG. 7, the pads 358 are illustrated as generally crescent moon shaped, having a first semi-circular side surface 360, a second semi-circular side surface 362, a convex surface 364 extending between the first side surface 360 and the second side surface 362, and a concave surface 366 extending between the first side surface 360 and the second side surface 362. The concave surface 366 may allow the pads 358 to conform to the contours of the epicardial surface of a heart when the connecting member 350 is positioned on a heart. It is noted that although the pads 358 are depicted as having a crescent moon shape, in other embodiments the pads 358 may assume any other desired shape, such as square, rectangular, circular, oval, polygonal, irregular, or the like.

In some embodiments, the concave surface 366 and/or the convex surface 364 of the pads 358 may include or be coated with a therapeutic agent, such as a therapeutic agent disclosed later herein. Thus, in some embodiments the surface of the pads 358 which is in contact with the epicardial surface of a heart may include or be coated with a therapeutic agent.

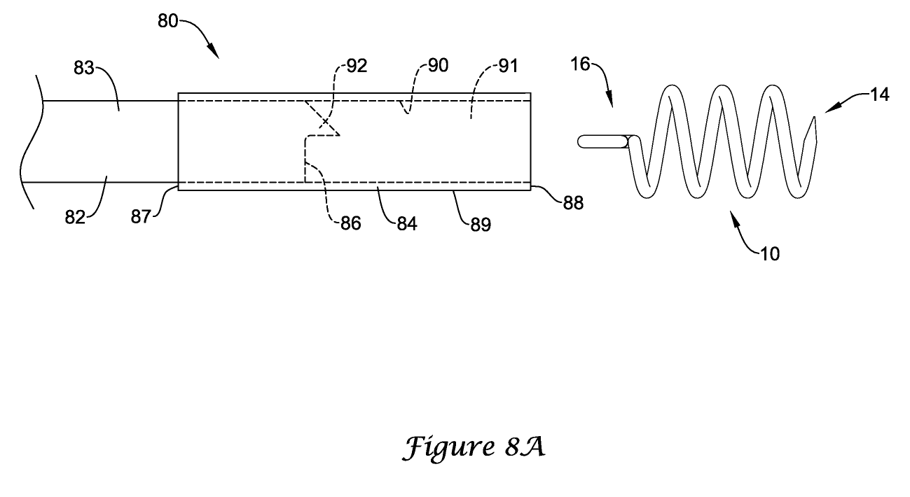

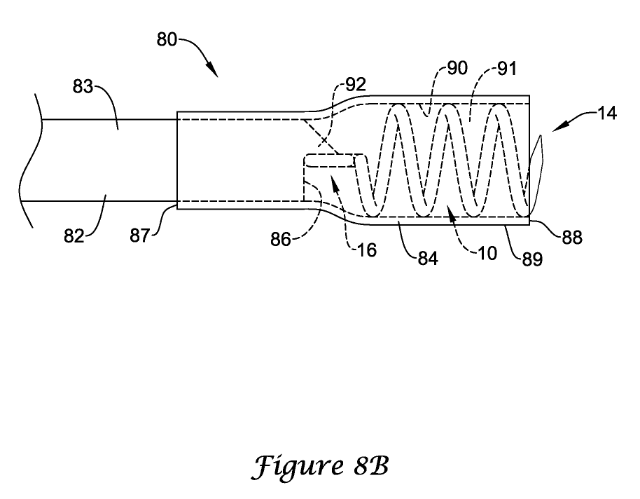

FIGS. 8A and 8B illustrate a tool 80 which may be used to secure a tissue anchor, such as the tissue anchor 10, to a wall of a heart. The tool 80 may include an elongate shaft 82. In some embodiments, the shaft 82 may be a tubular member, or the shaft 82 may be a solid member, having a solid cross-section. In some embodiments, the shaft 82 may be made of a polymeric material, or the shaft 82 may be formed of a metallic material. For example, in some embodiments the shaft 82 may be a hypotube.

A sleeve 84, such as a piece of heat-shrink tubing or other polymeric tubing, may extend over the distal end 86 of the shaft 82. Thus, the sleeve 84 may be positioned over a distal portion of the shaft 82. The sleeve 84 may be a tubular member having an annular wall including a first end 87, a second end 88, an outer peripheral surface 89, and an inner peripheral surface 90 defining a central opening 91 extending from the first end 87 to the second end 88 of the sleeve 84.

In some embodiments the sleeve 84 may be a piece of heat-shrink tubing which has been heat shrunk around the distal portion of the shaft 82, applying a radially inward compressive force onto the shaft 82. In other embodiments, the sleeve 84 may be a tube of another material such as another polymeric material. In some embodiments the sleeve 84 may be formed of a flexible or pliant material, such as silicone, rubber, an elastomeric polymer, a polymeric foam, or the like.

In some embodiments, the diameter of the central opening 91 of the sleeve 84 may be sized slightly smaller than the outer diameter of the portion of the shaft 82 in which the sleeve 84 is positioned over. Thus, in some embodiments, an interference or frictional fit may be established between the inner surface 90 of the sleeve 84 and the outer surface 83 of the shaft 82 in order to retain the sleeve 84 on the distal portion of the shaft 82. In some embodiments, the sleeve 84 may be adhered, bonded, crimped, threaded, or otherwise fastened to the distal portion of the shaft 82.

As shown in FIG. 8A, the distal end 88 of the sleeve 84 may extend distal of the distal end 86 of the shaft 82 beyond the distal end 86, while the proximal end 87 of the sleeve 84 may extend proximal of the distal end 86 of the shaft 82 over a portion of the shaft 82. Thus, a distal portion of the central opening 91 of the sleeve 84 may be unobstructed by the shaft 82, while a proximal portion of the central opening 91 of the sleeve 84 may be occupied by the shaft 82. In some embodiments, the distal end 86 of the shaft 82 may include a notch 92.

As shown in FIG. 8A, in some embodiments the notch 92 may be formed by a jog in the distal end 86 of the shaft 82. Thus, in such embodiments a portion of the distal end 86 of the shaft 82 may extend further distally than another portion of the distal end 86 of the shaft 82. As shown in FIG. 8B, the notch 92 may accommodate the eyelet 16 of a tissue anchor 10 loaded in the tool 80. Thus, an edge of the notch 92 may be in contact with the eyelet 16 of the anchor 10.

In some embodiments, the inner diameter of the sleeve 84 may be sized slightly smaller than the outer diameter of the helical portion 12 of the tissue anchor 10. During use the tissue anchor 10 may be loaded into the central opening 91 of the sleeve 84. Thus, in embodiments in which the inner diameter of the sleeve 84 is sized slightly less than the outer diameter of the helical portion 12 of the tissue anchor 10, when the tissue anchor 10 is loaded into the central opening 91 of the sleeve 84, an interference or frictional fit between the tissue anchor 10 and the sleeve 84 may retain the tissue anchor 10 in the opening 91.

As shown in FIG. 8B, with the tissue anchor 10 loaded into the central opening 91 of the sleeve 84, the sleeve 84 may expand slightly radially outward in order to accommodate the tissue anchor 10 in the opening 91 of the sleeve 84. Thus, the interference fit between the tissue anchor 10 and the sleeve 84 may result in the sleeve 84 being placed in tension, thereby applying an inward compressive force on the tissue anchor 10 to help retain the tissue anchor 10 within the opening 91. Although the radially inward compressive force may retain the tissue anchor 10 within the opening 91, the tissue anchor 10 may be removed from the sleeve 84 when desired. For example, once the tissue anchor 10 is secured to a wall of a heart, the shaft 82 and sleeve 84 may be pulled away from the tissue anchor 10, separating the tissue anchor 10 from the tool 80.

Also shown in FIG. 8B, the notch 92 of the shaft 82 is shown engaged with or in contact with the eyelet 16 of the tissue anchor 10. With the notch 92 of the shaft 82 engaged with the eyelet 16 of the tissue anchor 10, rotation of the shaft 82 may result in equivalent rotation of the tissue anchor 10. In other words, torsional forces transmitted through the shaft 82 may be directed to the tissue anchor 10 through the interface between the notch 92 and the eyelet 16 of the tissue anchor 10.

During operation, the tissue anchor 10 may be loaded into the sleeve 84 of the tool 80 such that a distal portion of the tissue anchor 10 including the tissue piercing tip 14 is exposed beyond the distal end 88 of the sleeve 84. The tissue piercing tip 14 may then be placed in contact with the epicardial surface of a heart and the shaft 82 may be rotated. Rotation of the shaft 82 in turn rotates the tissue anchor 10, causing the helical portion 12 of the tissue anchor 10 to be screwed into the wall of the heart. In some embodiments, the helical portion 12 may be screwed into the epicardium layer and/or myocardium layer of the heart, yet not extend into and/or through the endocardium layer of the heart. Once the tissue anchor 10 is properly anchored to the wall of the heart, the tool 80 may be removed, leaving the tissue anchor 10 embedded into the wall of the heart.

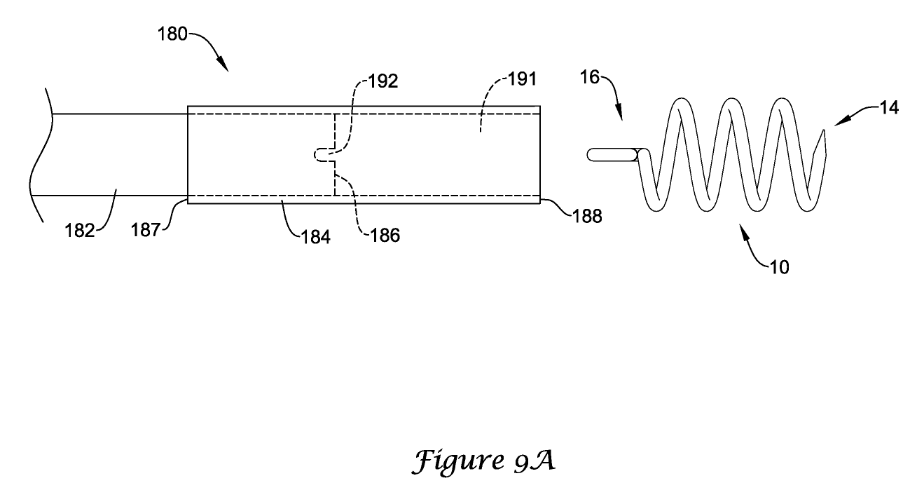

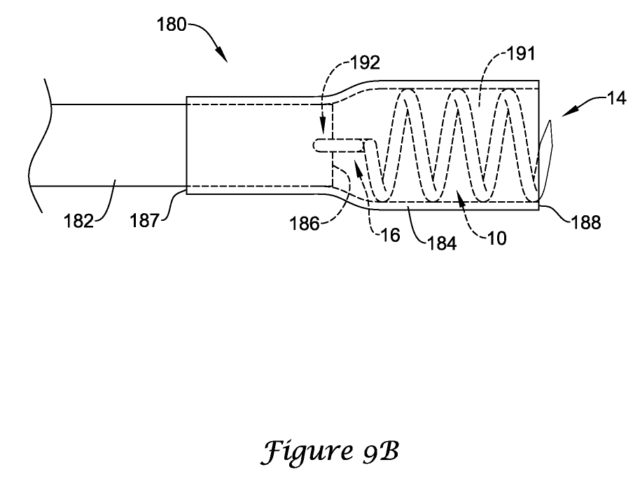

Another tool 180 which may be used to secure a tissue anchor, such as the tissue anchor 10, to a wall of a heart is shown in FIGS. 9A and 9B. The tool 180 may be similar to the tool 80, described above. Thus, in the interest of brevity, similarities in construction and operation of the tool 180 with those of the tool 80 will not be reiterated. For example, the tool 180 may include a shaft 182 and a sleeve 184 coupled to the shaft 182, such that the distal end 188 of the sleeve 184 extends beyond the distal end 186 of the shaft 182. Thus, the proximal end 187 of the sleeve 184 may be located proximal of the distal end 186 of the shaft 182, while the distal end 188 of the sleeve 184 may be located distal of the distal end 186 of the shaft 182. Thus, a distal portion of the central opening 191 of the sleeve 184 may be unobstructed by the shaft 182, while a proximal portion of the central opening 191 of the sleeve 184 may be occupied by the shaft 182.

As shown in FIG. 9A, a channel 192 may be formed in the distal end 186 of the shaft 182. The channel 192 may be a recess in the distal end 186 of the shaft 182, extending proximally from the distal end 186 of the shaft 182. As shown in FIG. 9B, the channel 192 may be sized to receive the eyelet 16 of the tissue anchor 10 when the tissue anchor 10 is loaded in the tool 180. In other words, the eyelet 16 of the tissue anchor 10 may extend into the channel 192 when the tissue anchor 10 is loaded into the sleeve 184 of the tool 180. With the eyelet 16 of the tissue anchor 10 engaged with the channel 192 of the shaft 182, rotation of the shaft 182 may result in equivalent rotation of the tissue anchor 10. In other words, torsional forces transmitted through the shaft 182 may be directed to the tissue anchor 10 through the interface between the channel 192 and the eyelet 16 of the tissue anchor 10.

As shown in FIG. 9B, with the tissue anchor 10 loaded into the central opening 191 of the sleeve 184, the sleeve 184 may expand slightly radially outward in order to accommodate the tissue anchor 10 in the opening 191 of the sleeve 184. Thus, the interference fit between the tissue anchor 10 and the sleeve 184 may result in the sleeve 184 being placed in tension, thereby applying an inward compressive force on the tissue anchor 10 to help retain the tissue anchor 10 within the opening 191. Although the radially inward compressive force may retain the tissue anchor 10 within the opening 191, the tissue anchor 10 may be removed from the sleeve 184 when desired. For example, once the tissue anchor 10 is secured to a wall of a heart, the shaft 182 and sleeve 184 may be pulled away from the tissue anchor 10, separating the tissue anchor 10 from the tool 180.

During operation, the tissue anchor 10 may be loaded into the sleeve 184 of the tool 180 such that a distal portion of the tissue anchor 10 including the tissue piercing tip 14 is exposed beyond the distal end 188 of the sleeve 184. The tissue piercing tip 14 may then be placed in contact with the epicardial surface of a heart and the shaft 182 may be rotated. Rotation of the shaft 182 in turn rotates the tissue anchor 10, causing the helical portion 12 of the tissue anchor 10 to be screwed into the wall of the heart. In some embodiments, the helical portion 12 may be screwed into the epicardium layer and/or myocardium layer of the heart, yet not extend into and/or through the endocardium layer of the heart. Once the tissue anchor 10 is properly anchored to the wall of the heart, the tool 180 may be removed, leaving the tissue anchor 10 embedded into the wall of the heart.

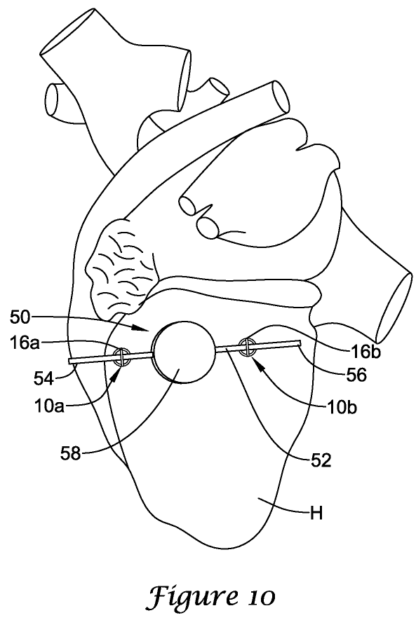

FIG. 10 shows one possible assembly including a connecting member and a pair of tissue anchors, as described above, positioned on the exterior of a heart H. As shown in FIG. 10, a first tissue anchor 10a may be anchored into the wall of the heart H from the epicardial surface of the heart H. A second tissue anchor 10b may be anchored into the wall of the heart H from the epicardial surface of the heart H at a short distance away from the first tissue anchor 10a.

The tissue anchors 10a/10b may be anchored to the wall of the heart H by rotating the tissue anchors 10a/10b by hand, or with a tool, such as one of the tools 80/180 described above. Rotation of the tissue anchors 10a/10b embeds the helical portion 12 of the tissue anchors 10a/10b into the wall of the heart H.

In some embodiments, a pledget (e.g., a flat, absorbent pad) may be placed between the tissue anchors 10a/10b and the epicardial surface of the heart prior to anchoring the tissue anchors 10a/10b into the wall of the heart H. In securing the tissue anchors 10a/10b to the heart wall, the tissue anchors 10a/10b may penetrate the pledgets prior to penetrating the epicardial surface of the heart H. The pledgets may help anchor the tissue anchors 10a/10b in place and/or stop any minor bleeding that may occur at the penetration site where the tissue anchors 10a/10b penetrate the epicardial surface of the heart H.

A connecting member 50 may be placed exterior of the heart H between the first tissue anchor 10a and the second tissue anchor 10b such that the connecting member 50 spans a portion of the heart H between the first tissue anchor 10a and the second tissue anchor 10b.

As shown in FIG. 10, the first end 54 of the shaft 52 of the connecting member 50 may be extended through the eyelet 16a of the first tissue anchor 10a and the second end 56 of the shaft 52 of the connecting member 50 may be extended through the eyelet 16b of the second tissue anchor 10b. In an embodiment in which the shaft 52 may include one or more notches such as the notches 170 shown in FIG. 5 along a portion of the shaft 152, the upper portion of the eyelet 16 may rest or be cradled in a notch of the shaft 52.

In some embodiments, an atraumatic cap (not shown) may be placed over one or more of the first end 54 and/or the second end 56 of the shaft 52 to reduce the possibility of the first end 54 and/or the second end 56 of the shaft 52 injuring anatomical tissue within the thoracic cavity. In some embodiments, the atraumatic cap may be a biocompatible polymeric cap extending over a portion of the first end 54 and/or the second end 56 of the shaft 52. In other embodiments, other means may be implemented in order to reduce any injury to anatomical tissue within the thoracic cavity.

A central portion of the connecting member 50, such as the pad 58 of the connecting member 50, may be positioned between the first tissue anchor 10a and the second tissue anchor 10b. The central portion or pad 58 may be in contact with the epicardial surface of the heart H, pushing inward on the epicardial surface of the heart H. The tissue anchors 10a/10b may counteract the inward force of the central portion or pad 58 pushing on the epicardial surface of the heart H.

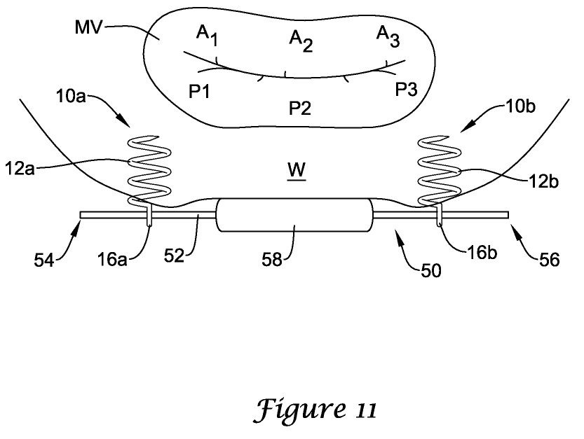

FIG. 11 is a view representing one possible placement of the components of the assembly as shown in FIG. 10, in relation to the mitral valve MV and wall W of a heart H. The mitral valve MV includes two leaflets, an anterior leaflet and a posterior leaflet. The anterior leaflet includes three scallops, described as A1, A2 and A3, and labeled as such in FIG. 11. The posterior leaflet includes three scallops, described as P1, P2 and P3, and labeled as such in FIG. 11. Thus, regions A1, A2, A3, P1 , P2 and P3 generally designate regions of the mitral valve associated with the identified scallops of the valve leaflets.

As shown in FIG. 11, in some embodiments the first tissue anchor 10a may be anchored to the wall W of the heart H at a location proximate the P1 region of the mitral valve MV. For example, in some embodiments the first tissue anchor 10a may be anchored to the wall W of the heart H at a location exterior of the P1 scallop of the posterior leaflet of the mitral valve MV. Additionally or alternatively, as shown in FIG. 11, in some embodiments the second tissue anchor 10b may be anchored to the wall W of the heart H at a location proximate the P3 region of the mitral valve MV. For example, in some embodiments the second tissue anchor 10b may be anchored to the wall W of the heart H at a location exterior of the P3 scallop of the posterior leaflet of the mitral valve MV. The pad 58 or central portion of the connecting member 50 may be positioned in contact with the wall W of the heart H proximate the P2 region of the mitral valve MV. For example, in some embodiments the pad 58 or central portion of the connecting member 50 may be positioned in contact with the wall W of the heart H exterior of the P2 scallop of the posterior leaflet of the mitral valve MV.

Thus, in some embodiments, the first tissue anchor 10a may be anchored to the wall W of the heart H at a location exterior of the P1 scallop of the posterior leaflet of the mitral valve MV, the anchoring location of the first tissue anchor 10a being closer to the P1 scallop than to either of the P2 or P3 scallops of the posterior leaflet. Likewise, in some embodiments the second tissue anchor 10b may be anchored to the wall W of the heart H at a location exterior of the P3 scallop of the posterior leaflet of the mitral valve MV, the anchoring location of the second tissue anchor 10a being closer to the P3 scallop than to either of the P1 or P2 scallops of the posterior leaflet. Furthermore, in some embodiments, the contact location of the pad 58 or central portion of the connecting member 50 with the epicardial surface of the heart H may be closer to the P2 scallop of the posterior leaflet of the mitral valve than to either of the P1 or P3 scallops of the posterior leaflet.

In other embodiments, the tissue anchors 10a/10b and/or the pad 58 or central portion of the connecting member 50 may be positioned at other locations in relation to the regions of the mitral valve of the heart. For example, in some embodiments the pad 58 or central portion of the connecting member 50 may be placed directly exterior of the P1 region of the mitral valve or directly exterior of the P3 region of the mitral valve in order to provide an inward force at the P1 region or P3 region of the mitral valve, respectively. In such embodiments, the location of the tissue anchors 10a/10b would be shifted to either side of the P1 region or the P3 region, respectively, such that the pad 58 or central portion of the connecting member 50 may be centrally located between the tissue anchors 10a/10b. Thus, in some embodiments, the pad 58 or central portion of the connecting member 50 may be placed directly exterior of the P1 scallop or the P3 scallop of the posterior leaflet of the mitral valve MV. Therefore, in some embodiments the contact location of the pad 58 or central portion of the connecting member 50 with the epicardial surface of the heart H may be closer to the P1 scallop of the posterior leaflet of the mitral valve than to either of the P2 or P3 scallops of the posterior leaflet, or the contact location of the pad 58 or central portion of the connecting member 50 with the epicardial surface of the heart H may be closer to the P3 scallop of the posterior leaflet of the mitral valve than to either of the P1 or P2 scallops of the posterior leaflet.

In other embodiments, one or both of the tissue anchors 10a/10b may be secured to an epicardial surface of the heart H in the right ventricular wall of the heart H. In such embodiments, the pad 58 or central portion of the connecting member 50 may be placed directly exterior of the P1 scallop, the P2 scallop, the P3 scallop, or at another epicardial location of the heart H to achieve a desired reshaping of the mitral valve. For example, in some embodiments the contact location of the pad 58 may be located between the P1 scallop and the P2 scallop, or between the P2 scallop and the P3 scallop.

The inward force exerted on the epicardial surface of the heart wall by the pad 58 or central portion of the connecting member 50 and/or the outward forces exerted on the heart wall by the tissue anchors 10a/10b may contribute to remodeling the anatomical structure of the mitral valve, such as reshaping of the annulus of the mitral valve. For instance, in some embodiments, the inward force exerted on the epicardial surface of the heart wall by the pad 58 or central portion of the connecting member 50 may help restore coaptation of the anterior and posterior leaflets of the mitral valve by reducing the septal-lateral distance and/or anterior-posterior distance between the leaflets. Improving the coaptation of the leaflets of the mitral valve, may reduce and/or eliminate mitral regurgitation of the patient.

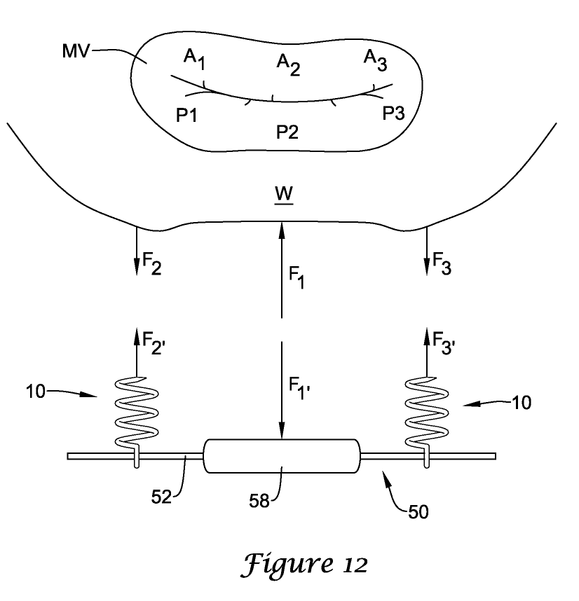

FIG. 12 is a force diagram showing the forces, depicted as vectors, exerted on various portions of the heart H and components of the assembly in the configuration shown in FIG. 11. A vector is a quantity that has both magnitude and direction. Thus, the force vectors illustrated in FIG. 12 depict both the magnitude and direction of forces acting on the various portions of the heart H and components of the shaping assembly. Such magnitudes and directions of forces may be achieved when the shaft 52 of the connecting member 50 is a rigid, non-flexible shaft 52.

As shown in FIG. 12, the pad 58 of the connecting member 50 may exert an inward force F₁ on the wall W of the heart H. As used herein, the term “inward force” is intended to mean a force having a direction directed generally inward toward the interior of the heart, such as generally toward the mitral valve MV from the surface of the heart H. Additionally, each of the pair of tissue anchors 10a/10b may exert an outward force F₂/F₃ on the wall W of the heart H. As used herein, the term “outward force” is intended to mean a force having a direction directed generally outward away from the interior of the heart, such as generally away from the mitral valve from the surface of the heart H. Not inconsistent with the forces exerted onto the wall W of the heart H, counter forces may be exerted onto the connecting member 50 and/or the tissue anchors 10a/10b. For instance, an outward force F₁′ may be exerted on the pad 58 of the connecting member 50, and inward forces F₂′/F₃′ may be exerted on each of the tissue anchors 10a/10b.

The outward force F₁′ acting on the connecting member 50 may have a magnitude equal to the magnitude of the inward force F₁ acting on the heart wall W, and the outward force F₁′ acting on the connecting member 50 may have a direction opposite the direction of the inward force F₁ acting on the heart wall W. The inward force F₂′ acting on the tissue anchor 10a may have a magnitude equal to the magnitude of the outward force F₂ acting on the heart wall W, and the inward force F₂′ acting on the tissue anchor 10a may have a direction opposite the direction of the outward force F₂ acting on the heart wall W. The inward force F₃′ acting on the tissue anchor 10b may have a magnitude equal to the magnitude of the outward force F₃ acting on the heart wall, and the inward force F₃′ acting on the tissue anchor 10b may have a direction opposite the direction of the outward force F₃ acting on the heart wall W.

In some embodiments, the direction of the outward force F₂ may be opposite the direction of the inward force F₁. Additionally, in some embodiments the direction of the outward force F₃ may be opposite the direction of the inward force F₁. In such embodiments, the direction of the inward forces F₂′ and F₃′ would be opposite the direction of the outward force F₁′.

In some embodiments, the sum of the magnitudes of the outward forces F₂/F₃ acting on the heart wall W may be equal to the magnitude of the inward force F₁ acting on the heart wall W. For instance, in some embodiments, the magnitude of each of the outward forces F₂/F₃ may be one-half of the magnitude of the inward force F₁. Additionally, in some embodiments the sum of the magnitudes of the inward forces F₂′/F₃′ acting on the connecting member 50 may be equal to the magnitude of the outward force F₁′ acting on the connecting member 50. For instance, in some embodiments, the magnitude of each of the inward forces F₂′/F₃′ may be one-half of the magnitude of the outward force F₁′.

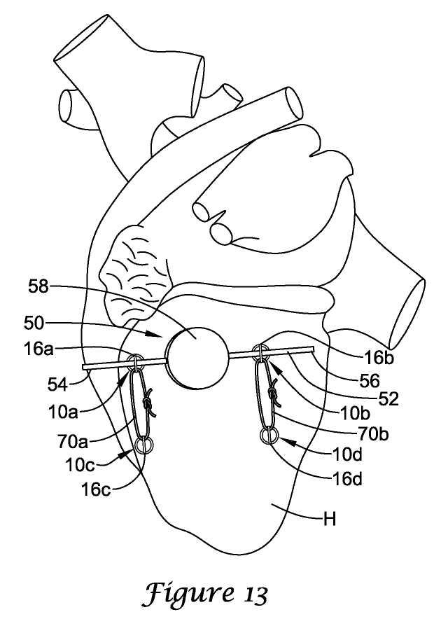

FIG. 13 shows another possible assembly including a connecting member and two pairs of tissue anchors, as described above, positioned on the exterior of a heart. The arrangement of the connecting member 50 and the first and second tissue anchors 10a/10b may be similar to the arrangement discussed in relation to that depicted in FIGS. 10 and 11.

As shown in FIG. 13, a first tissue anchor 10a may be anchored into the wall of the heart H from the epicardial surface of the heart H. A second tissue anchor 10b may be anchored into the wall of the heart H from the epicardial surface of the heart H at a short distance away from the first tissue anchor 10. A connecting member 50 may be placed exterior of the heart H between the first tissue anchor 10a and the second tissue anchor 10b such that the connecting member 50 spans a portion of the heart H between the first tissue anchor 10a and the second tissue anchor 10b.

As shown in FIG. 13, the first end 54 of the shaft 52 of the connecting member 50 may be extended through the eyelet 16a of the first tissue anchor 10a and the second end 56 of the shaft 52 of the connecting member 50 may be extended through the eyelet 16b of the second tissue anchor 10b. In an embodiment in which the shaft 52 may include one or more notches, such as the notches 170 shown in FIG. 5 along a portion of the shaft 152, the upper portion of the eyelet 16 may rest or be cradled in a notch of the shaft 52.

The pad 58 or central portion of the connecting member 50 may be positioned between the first tissue anchor 10a and the second tissue anchor 10b. The pad 58 or central portion may be in contact with the epicardial surface of the heart H, pushing inward on the epicardial surface of the heart H. The tissue anchors 10a/10b may counteract the inward force of the pad 58 or central portion pushing on the epicardial surface of the heart H.

Additionally, as shown in FIG. 13, a third tissue anchor 10c and a fourth tissue anchor 10d may be anchored to the wall of the heart H. The third tissue anchor 10c may be positioned at a location inferior to the first tissue anchor 10a, and the fourth tissue anchor 10d may be positioned at a location inferior to the second tissue anchor 10d. In some embodiments, the third tissue anchor 10c may be positioned laterally to the fourth tissue anchor 10d.

The third tissue anchor 10c may be anchored to the wall of the heart H, such as the ventricular wall of the heart H, exterior of the papillary muscles of the left ventricle. The fourth tissue anchor 10d may be anchored to the wall of the heart H, such as the ventricular wall of the heart H, exterior of the papillary muscles of the left ventricle.

A first member, such as a suture 70, may extend between the first anchor 10a and the third anchor 10c. Although the first member is shown as a suture 70, in other embodiments, the first member may be a rigid element, such as a clip or other member, extending between the first anchor 10a and the third anchor 10c. As shown in FIG. 13, the first suture 70a may be placed through the eyelets 16a/16c of the anchors 10a/10c and securely knotted. Connecting the third anchor 10c to the first anchor 10a may draw the third anchor 10c upward toward the first anchor 10a. Drawing the third anchor 10c upward may reposition the papillary muscles in the left ventricle, such as move the papillary muscles in the left ventricle back to or toward their natural position, relieving stress on the chordae tendinae extending between the papillary muscles and the leaflets of the mitral valve. Over time, the reduced stress in the ventricle of the heart H may allow the heart H to be remodeled to a pre-diseased state.

Additionally or alternatively, a second member, such as a suture 70 may extend between the second anchor 10b and the fourth anchor 10d. Although the second member is shown as a suture 70, in other embodiments, the second member may be a rigid element, such as a clip or other member, extending between the second anchor 10b and the fourth anchor 10d. As shown in FIG. 13, the second suture 70b may be placed through the eyelets 16b/16d of the anchors 10b/10d and securely knotted. Connecting the fourth anchor 10d to the second anchor 10b may draw the fourth anchor 10d upward toward the second anchor 10b. Drawing the fourth anchor 10d upward may reposition the papillary muscles in the left ventricle, such as move the papillary muscles in the left ventricle back to or toward their natural position, relieving stress on the chordae tendinae extending between the papillary muscles and the leaflets of the mitral valve. Over time, the reduced stress in the ventricle of the heart H may allow the heart H to be remodeled to a pre-diseased state.

Furthermore, drawing upward on the wall of the heart exterior of the papillary muscles in the left ventricle with the third and/or fourth tissue anchors 10c/10d may additionally and/or alternatively improve coaptation of the leaflets of the mitral valve in order to reduce or eliminate retrograde flow of blood from the left ventricle through the mitral valve and into the left atrium. For instance, drawing upward on the wall of the heart exterior of the papillary muscles in the left ventricle may reduce the distance between the locations in which the chordae tendinae are attached between the papillary muscles and a leaflet of the mitral valve. Reducing this distance may improve coaptation of the valve leaflets, and thus reduce mitral regurgitation.

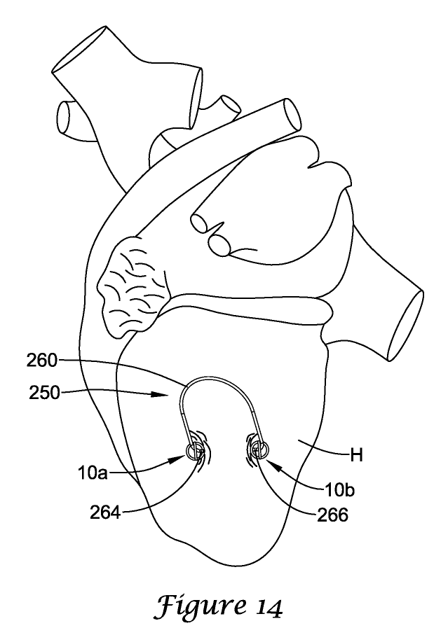

FIG. 14 shows another possible assembly including a connecting member and a pair of tissue anchors, as described above, positioned on the exterior of a heart. As shown in FIG. 14, a first anchor 10a may be anchored into the wall of the heart H from the epicardial surface of the heart H. A second tissue anchor 10b may be anchored into the wall of the heart H from the epicardial surface of the heart H at a short distance away from the first tissue anchor 10.

The tissue anchors 10a/10b may be anchored to the wall of the heart H by rotating the tissue anchors 10a/10b by hand, or with a tool, such as one of the tools 80/180 described above. Rotation of the tissue anchors 10a/10b embeds the helical portion 12 of the tissue anchors 10a/10b into the wall of the heart H.

A connecting member 250 may be placed exterior of the heart H between the first tissue anchor 10a and the second tissue anchor 10b such that the connecting member 250 is attached to the first tissue anchor 10a and the second tissue anchor 10b.

As shown in FIG. 10, the first stub 264 proximate the first end 254 of the shaft 252 of the connecting member 250 may be extended through the eyelet 16a of the first tissue anchor 10a and the second stub 266 proximate the second end 256 of the shaft 252 of the connecting member 250 may be extended through the eyelet 16b of the second tissue anchor 10b. In an embodiment in which the shaft 252 may include one or more notches, such as the notches 170 shown in FIG. 5 along a portion of the shaft 152, the upper portion of the eyelet 16 may rest or be cradled in a notch of the shaft 252.

The U-shaped curvature portion 260 of the shaft 252 is shown extending around a portion of the exterior of the heart H. The shaft 252, which may be a rigid, non-flexible member, may apply inward forces on the wall of the heart H at the locations of the tissue anchors 10a/10b in order to reshape or remodel a portion of the heart.

The inward forces applied to the wall of the heart H exterior of the papillary muscles in the left ventricle with the connecting member 250 may improve coaptation of the leaflets of the mitral valve in order to reduce or eliminate retrograde flow of blood from the left ventricle through the mitral valve and into the left atrium. For example, inward forces at the locations of the first and second anchors 10a/10b may push the papillary muscles closer to the mitral valve, reducing the distance between the papillary muscles and the leaflets of the mitral valve, and thus relieving stress on the chordae tendinae extending between the papillary muscles and the leaflets of the mitral valve. By relieving stresses on the chordae tendinae, the connecting member 250 and tissue anchors 10a/10b of the assembly placed on the heart H may improve coaptation of the leaflets of the mitral valve.

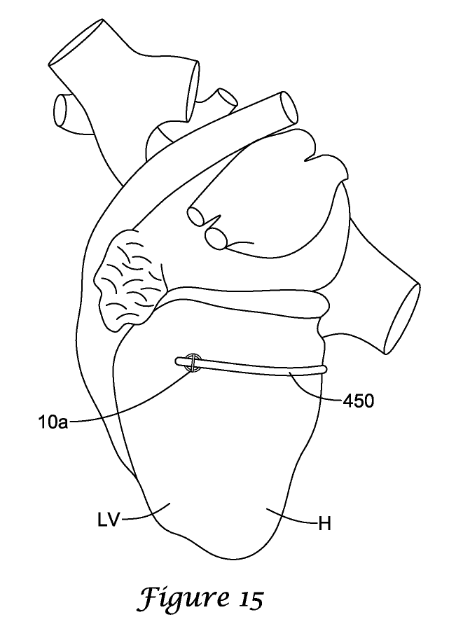

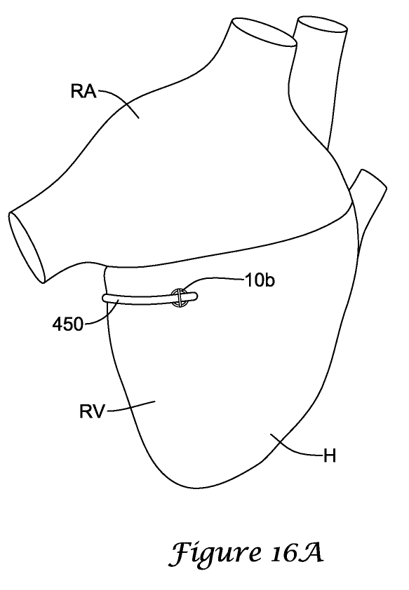

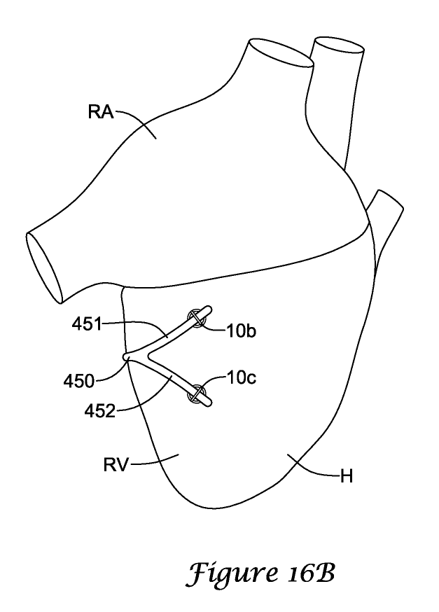

FIGS. 15, 16A and 16B show yet another possible assembly including a connecting member and a plurality of tissue anchors, as described above, positioned on the exterior of a heart. As shown in FIG. 15, a first tissue anchor 10a may be anchored into the wall of the heart H from the epicardial surface of the heart H. For example, the first tissue anchor 10a may be anchored into the wall of the left ventricle LV proximate the P2 region of the mitral valve. In other embodiments, the first tissue anchor 10a may be anchored into the wall of the left ventricle LV proximate the P1 region or the P3 region of the mitral valve, exterior of the papillary muscles, or at another location on the left ventricular wall, if desired.

FIGS. 16A and 16B, show two possible arrangements of the assembly on the opposite side of the heart H. As shown in FIG. 16A, a second tissue anchor 10b may be anchored into the wall of the right ventricle RV from the epicardial surface of the heart H at a location approximately polar opposite to the location of the first tissue anchor 10a.

A connecting member 450 may be placed exterior of the heart H between the first tissue anchor 10a and the second tissue anchor 10b. In some embodiments, the connecting member 450 may be a substantially C-shaped or U-shaped rigid member, such that the connecting member 450 may extend around a portion of the heart H between the first tissue anchor 10a and the second tissue anchor 10b. As shown in the drawings, the connecting member 450 may extend around a lateral side of the heart H from the left ventricle LV to the right ventricle RV. However, in other embodiments, the connecting member 450 may extend around a superior side or an inferior side of the heart from the left ventricle LV to the right ventricle RV, if desired.

The connecting member 450 may be sized and shaped such that the connecting member 450 may apply a force to the heart H through the first tissue anchor 10a and the second tissue anchor 10b. For example, the placement of the connecting member 450 between the first tissue anchor 10a and the second tissue anchor 10b may exert an inward force on the heart H at the location of the first tissue anchor 10a (e.g., P1 region, P2 region, P3 region, papillary muscles of the left ventricle) and an opposing inward force on the heart H at the location of the second tissue anchor 10b (e.g., right ventricular wall). The locations of the first tissue anchor 10a and the second tissue anchor 10b may allow the force exerted on the heart H by the first tissue anchor 10a to be generally opposed by the force exerted on the heart H by the second tissue anchor 10b.

As shown in FIG. 16B, an alternative arrangement may include two or more tissue anchors anchored into the epicardial surface of the right ventricle RV generally opposite to the first tissue anchor 10a. For example, a second tissue anchor 10b and a third tissue anchor 10c may be anchored into the wall of the right ventricle RV. The inclusion of two or more tissue anchors 10b/10c in the right ventricle RV may be advantageous to help distribute the counterforce opposing the force exerted on the wall of the heart H through the first tissue anchor 10a between two or more tissue anchors in the right ventricle RV.

In such an embodiment, the connecting member 450 may be directly or indirectly connected to two or more tissue anchors 10b/10c on the right ventricle RV. For example, as shown in FIG. 16B, the connecting member 450 may be a bifurcated connecting member including a first branch 451 extending to the second tissue anchor 10b and a second branch 452 extending to the third tissue anchor 10c. In other embodiments, an additional member may extend between the second tissue anchor 10b and the third tissue anchor 10c, wherein the connecting member 450 is connected to the additional member.

The connecting member 450 may apply an inward force on the walls of the heart H, urging the first tissue anchor 10a on the wall of the left ventricle LV toward the second tissue anchor 10b and/or third tissue anchor 10c on the wall of the right ventricle RV. The inward forces applied to the wall of the heart H with the connecting member 450 may improve coaptation of the leaflets of the mitral valve in order to reduce or eliminate retrograde flow of blood from the left ventricle through the mitral valve and into the left atrium. For example, inward forces at the locations of the first and second anchors 10a/10b may alter the anterior-posterior dimension or the septal-lateral dimension of the mitral valve. By altering the shape of the mitral valve, the connecting member 450 and tissue anchors 10a/10b of the assembly placed on the heart H may improve coaptation of the leaflets of the mitral valve. In other embodiments, the force exerted on the heart H by the connecting member 450 may be an outwardly directed force at the locations of the tissue anchors 10a/10b/10c.

In some embodiments, one or more components as described herein may include a drug eluting coating. The drug eluting coating may a controlled release of a therapeutic agent over a specified period of time. The therapeutic agent may be any medicinal agent which may provide a desired effect. Suitable therapeutic agents include drugs, genetic materials, and biological materials. Some suitable therapeutic agents which may be loaded in the drug eluting coating include, but are not necessarily limited to, antibiotics, antimicrobials, antioxidants, anti-arrhythmics, cell growth factors, immunosuppressants such as tacrolimus, everolimus, and rapamycin (sirolimus), therapeutic antibodies, wound healing agents, therapeutic gene transfer constructs, peptides, proteins, extracellular matrix components, steroidal and non-steroidal anti-inflammatory agents, anti-proliferative agents such as steroids, vitamins and restenosis inhibiting drugs, such as Taxol®, paclitaxel (i.e., paclitaxel, paclitaxel analogues, or paclitaxel derivatives, and mixtures thereof).

Although the above discussion is directed to improving the functioning of the mitral valve of a heart, one of skill in the art, incited by the present disclosure, would understand that the disclosed devices, assemblies and methods may be equally applicable to improving the functioning of another valve of a heart, such as the bicuspid valve, the aortic valve, or the pulmonary valve.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A method of improving the apposition of the valve leaflets of the mitral valve of a heart, the method comprising:
providing a first tissue anchor, a second tissue anchor, and a bar having a first end region, a second end region, and a central region between the first end region and the second end region;
anchoring the first tissue anchor into a wall of the heart from an epicardial surface of the heart;
anchoring the second tissue anchor into a wall of the heart from the epicardial surface of the heart; and
extending the bar between the first tissue anchor and the second tissue anchor such that the first end region of the rigid bar is attached to the first tissue anchor and the second end region of the rigid bar is attached to the second tissue anchor, the bar lying exterior of the epicardial surface of the heart;
wherein placement of the bar between the first tissue anchor and the second tissue anchor causes the central region to apply an inward first force on the epicardial surface of the heart, the inward first force represented by a vector having a magnitude and direction; and
wherein a pair of opposing outward second forces act on the heart wall by the first tissue anchor and the second tissue anchor, wherein each of the pair of opposing outward second forces are represented by a vector having a magnitude and direction, wherein the sum of the magnitudes of the vectors of the pair of outward second forces is equal to the magnitude of the inward first force, and wherein the direction of each of the vectors of the pair of outward second forces is opposite the direction of the inward first force.

2. The method of claim 1, wherein the direction of the inward force intersects the P2 region of the mitral valve.

3. The method of claim 2, wherein the first tissue anchor is secured to the myocardium of the heart at the P1 region of the mitral valve.

4. The method of claim 3, wherein the direction of the vector of the outward force acting on the heart wall by the first tissue anchor intersects the P1 region of the mitral valve.

5. The method of claim 4, wherein the second tissue anchor is secured to the myocardium of the heart at the P3 region of the mitral valve.

6. The method of claim 5, wherein the direction of the vector of the outward force acting on the heart wall by the second tissue anchor intersects the P3 region of the mitral valve.

7. The method of claim 1, wherein the central region of the bar includes a pad in contact with the epicardial surface of the heart.

8. A method of improving the functioning of the mitral valve of a heart, the method comprising:
providing a first corkscrew anchor having an eyelet and a second corkscrew anchor having an eyelet;
providing a member having a first end region, a second end region and a central region between the first end region and the second end region, wherein the member is able to be placed in compression;
securing the first corkscrew anchor into a wall of the heart from an epicardial surface of the heart;
securing the second corkscrew anchor into a wall of the heart from the epicardial surface of the heart;
placing the first end region of the member through the eyelet of the first corkscrew anchor; and
placing the second end region of the member through the eyelet of the second corkscrew anchor;
wherein placement of the member between the first corkscrew anchor and the second corkscrew anchor causes the central region of the rigid member to apply an inward first force on the epicardial surface of the heart, and wherein each of the first corkscrew anchor and the second corkscrew anchor apply an opposing outward second force on the wall of the heart.

9. The method of claim 8, wherein the inward force is represented by a vector having a magnitude and direction;
wherein each of the opposing outward forces are represented by a vector having a magnitude and direction; and
wherein the sum of the magnitudes of the vectors of the outward forces is equal to the magnitude of the inward force, and wherein the direction of each of the vectors of the outward forces is opposite the direction of the inward force.

10. The method of claim 9, wherein the heart includes a mitral valve and a P1 region, a P2 region and a P3 region associated with the mitral valve of the heart, wherein the direction of the vector of the outward force acting on the heart wall by the first corkscrew anchor intersects the P1 region of the mitral valve.

11. The method of claim 9, wherein the heart includes a mitral valve and a P1 region, a P2 region and a P3 region associated with the mitral valve of the heart, wherein the direction of the vector of the outward force acting on the heart wall by the second corkscrew anchor intersects the P3 region of the mitral valve.

12. The method of claim 9, wherein the heart includes a mitral valve and a P1 region, a P2 region and a P3 region associated with the mitral valve of the heart, wherein the direction of the inward force intersects the P2 region of the mitral valve.

13. The method of claim 8, wherein the first end region of the member includes one or more notches and the second end region of the member includes one or more notches; and
wherein the eyelet of the first corkscrew anchor is positioned in a notch of the first end region of the member and the eyelet of the second corkscrew anchor is positioned in a notch of the second end region of the member.

14. A method of reshaping the mitral valve of a heart, wherein the heart includes a P1 region, a P2 region and a P3 region associated with the mitral valve of the heart, the method comprising:
providing a first anchor, a second anchor, and a rigid bar having a first end region, a second end region, and a central region between the first end region and the second end region;
anchoring the first anchor into a wall of the heart from an epicardial surface of the heart at the P1 region of the heart;
anchoring the second anchor into a wall of the heart from the epicardial surface of the heart at the P3 region of the heart; and
extending the rigid bar between the first anchor and the second anchor such that the first end region of the rigid bar is attached to the first anchor and the second end region of the rigid bar is attached to the second anchor, the rigid bar lying exterior of the epicardial surface of the heart;
wherein placement of the rigid bar between the first anchor and the second anchor causes the central region to apply an inward force on the epicardial surface of the heart at the P2 region of the heart; and
wherein each of the first anchor and the second anchor apply an opposing outward force on the wall of the heart.

15. The method of claim 14, wherein the inward force is represented by a vector having a magnitude and direction;
wherein each of the opposing outward forces are represented by a vector having a magnitude and direction; and
wherein the sum of the magnitudes of the vectors of the outward forces is equal to the magnitude of the inward force, and wherein the direction of each of the vectors of the outward forces is opposite the direction of the inward force.

16. The method of claim 14, further comprising the step of anchoring a third anchor into a wall of the heart from the epicardial surface of the heart at a location inferior of the first anchor.

17. The method of claim 16, further comprising the step of securing a first suture between the first anchor and the third second anchor.

18. The method of claim 17, further comprising the step of tightening the first suture in order to reposition the papillary muscles of the heart.

19. The method of claim 16, further comprising the step of anchoring a fourth anchor into a wall of the heart from the epicardial surface of the heart at a location inferior of the second anchor.

20. The method of claim 19, further comprising the steps of securing a first suture between the first anchor and the third anchor and securing a second suture between the second anchor and the fourth anchor.

21. The method of claim 20, further comprising the steps of tightening the first suture and tightening the second suture in order to reposition the papillary muscles of the heart.

22. An assembly for use in improving the apposition of the valve leaflets of the mitral valve of a heart, the assembly including:
a first tissue anchor configured to be anchored into a wall of the heart from an epicardial surface of the heart at a first position;
a second tissue anchor configured to be anchored into a wall of the heart from the epicardial surface of the heart at a second position; and
a rigid connecting member configured to extend between the first and second tissue anchors exterior of the epicardial surface of the heart.

23. The assembly of claim 22, wherein the rigid connecting member is configured to apply an inward force on the epicardial surface of the heart.

24. The assembly of claim 22, wherein the first tissue anchor includes an eyelet and the second tissue anchor includes an eyelet, wherein a first end of the rigid connecting member is configured to extend through the eyelet of the first tissue anchor and a second end of the rigid connecting member is configured to extend through the eyelet of the second tissue anchor.

25. A kit for use in a medical procedure to improve the apposition of the valve leaflets of the mitral valve of a heart, the kit including:
a first corkscrew anchor having an eyelet, the first corkscrew anchor configured to be screwed into a wall of the heart from an epicardial surface of the heart at a first position;
a second corkscrew anchor having an eyelet, the second corkscrew anchor configured to be screwed into a wall of the heart from the epicardial surface of the heart at a second position;
a rigid connecting member configured to extend between the first corkscrew anchor and the second corkscrew anchor exterior of the epicardial surface of the heart, the rigid connecting member including a first end configured to extend through the eyelet of the first corkscrew anchor and a second end configured to extend through the eyelet of the second corkscrew anchor; and
a tool configured to screw the first and second corkscrew anchors into a wall of the heart, the tool including an elongate shaft having a distal end and a tubular sleeve extending distally of the distal end of the elongate shaft, the sleeve configured to releasably retain a corkscrew anchor loaded within a lumen of the sleeve.

26. The kit of claim 25, wherein the rigid connecting member is configured to apply an inward force on the epicardial surface of the heart.

27. The kit of claim 25, wherein the tubular sleeve has an inner diameter and the first and second corkscrew anchors have an outer diameter greater than the inner diameter of the tubular sleeve.

28. A method of reshaping the mitral valve of a heart, the method comprising:
anchoring a first anchor into a wall of the heart from an epicardial surface of the heart;
anchoring a second anchor into a wall of the heart from the epicardial surface of the heart at a location inferior of the first anchor;
securing a first suture between the first anchor and the second anchor; and
tightening the first suture in order to reposition the papillary muscles of the heart.

29. The method of claim 28, wherein the first anchor is located exterior of the mitral valve and the second anchor is anchored into a left ventricular wall of the heart.

30. The method of claim 28, further comprising the steps of:
anchoring a third anchor into a wall of the heart from the epicardial surface of the heart at a location generally lateral to the first anchor;
anchoring a fourth anchor into a wall of the heart from the epicardial surface of the heart at a location inferior to the third anchor;
securing a second suture between the third anchor and the fourth anchor; and
tightening the second suture in order to reposition the papillary muscles of the heart.

31. The method of claim 30, wherein the heart includes a P1 region, a P2 region and a P3 region associated with the mitral valve of the heart, wherein the first anchor is located at the P1 region of the heart and third anchor is located at the P3 region of the heart.

32. The method of claim 30, further comprising:
extending a rigid member between the first anchor and the third anchor such that the rigid member applies an inward force on the epicardial surface of the heart.

33. The method of claim 32, wherein the heart includes a P1 region, a P2 region and a P3 region associated with the mitral valve of the heart, wherein the inward force is applied at the P2 region of the heart.
